(19) United States
(12) Patent Application Publication
(10) Pub. No.: US 2005/0172962 A1
(43) Pub. Date: Aug. 11, 2005

(54) Blister pack for use with an inhalation device

(75) Inventors: Anand V. Gumaste, West Windsor, NJ (US); John Bowers, Clarksburg, NJ (US)

Correspondence Address: Norman P. Soloway;HAYES SOLOWAY P.C., 130 W. Cushing Street, Tucson, AZ, 85701, US

(73) Assignee: MICRODOSE TECHNOLOGIES, INC.

(21) Appl. No.: 11/050,248

(22) Filed: Feb. 3, 2005

Related U.S. Application Data
(60) Provisional application No. 60/542,368, filed on Feb. 6, 2004.

(51) Int. Cl. B05D 7/14; A61M 15/00

(52) U.S. Cl. 128/203.15; 128/203.21

(57) ABSTRACT

An inhalation device is described for use with a medication pack wherein the medication pack is formed by a single sheet folded on itself into several sealed pockets or pleats. Each of the pockets or pleats contains a drug to be aerosolized using a piezoelectric element. The inhaler includes a mechanism to open the blisters by pulling apart the sealed pocket or pleat.

### CROSS REFERENCE TO RELATED APPLICATION

This application claims priority from U.S. Provisional Application Ser. No. 60/542,368, filed Feb. 6, 2004.

### TECHNICAL FIELD

The present invention relates generally to the field of inhalation devices, and more specifically, to inhalation devices that utilize vibration to facilitate suspension of medication into an inhaled gas stream (e.g., of inhaled air), and to medication blister packs for use therewith.

Particular utility for the present invention is found in the area of facilitating inhalation of powdered medications (e.g., bacterial vaccines, sinusitis vaccines, antihistaminic agents, vaso-constricting agents, anti-bacterial agents, anti-asthmatic agents, theophylline, aminophylline, di-sodium cromolyn, etc.), although other utilities, including other medicament applications such as facilitating inhalation of other powdered materials and/or liquid droplets, e.g. of insulin, vitamins, etc., are contemplated.

### BACKGROUND OF THE INVENTION

Certain diseases of the respiratory tract are known to respond to treatment by the direct application of therapeutic agents. As many of these agents are most readily available in dry powdered form, their application is most conveniently accomplished by inhaling the powdered material through the nose or mouth. This powdered form results in the better utilization of the medication in that the drug may be deposited exactly at the site desired and where its action may be required; hence, very minute doses of the drug are often equally as efficacious as larger doses administered by other means, with a consequent marked reduction in the incidence of undesired side effects and medication cost. Alternatively, the drug in this form may be used for treatment of diseases other than those of the respiratory system. When the drug is deposited on the very large surface areas of the lungs, it may very rapidly be absorbed into the blood stream; hence, this method of application may take the place of administration by injection, tablet, or other conventional means.

It is the opinion of the pharmaceutical industry that the bioavailability of the drug is optimum when the drug particles delivered to the respiratory tract are between about 1 to 5 microns in size. For delivering drug particles in this size range, a dry powder delivery system needs to address a number of issues:

First, small size particles develop an electrostatic charge on themselves during manufacturing and storage. This may cause the particles to agglomerate or aggregate, resulting in clusters of particles, which have an effective size greater than about 5 microns. The probability of these large clusters making it to the deep lungs then decreases. This in turn results in a lower percentage of the packaged drug being available to the patient for absorption.

Secondly, the dosage amount of active drug that needs to be delivered to the patient may be of the order of 10s of micrograms. For example, albuterol, in the case of a drug used by patients suffering from asthma, the dosage amount is usually about 25 to 50 micrograms. Current manufacturing equipment can effectively deliver aliquots of drugs in milligram dose range with acceptable accuracy. Therefore, the standard practice is to mix the active drug with a filler or bulking agent such as lactose. This additive also makes the drug “easy to flow.” This filler is also called a carrier since the drug particles also stick to these particles through electrostatic or chemical bonds. However, these carrier particles are very much larger than the drug particles in size. Thus, the ability of the dry powder inhaler to separate drug from the carrier is an important performance parameter in the effectiveness of the design.

Finally, active drug particles with sizes greater than about 5 microns typically will be deposited either in the mouth or throat. This introduces another level of uncertainty since the bioavailability and absorption of the drug in these locations generally is different from the lungs. Dry powder inhalers need to minimize the drug deposited in the mouth or throat to reduce the uncertainty associated with the bioavailability of the drug.

Prior art dry powder inhalers (DPIs) usually have a means for introducing the drug (active drug plus carrier) into a high velocity air stream. The high velocity air-stream is used as the primary mechanism for breaking up the clusters of micronized particles or separating the drug particles from the carrier. Several inhalation devices useful for dispensing this powder form of medication are known in the prior art. For example, in U.S. Pat. Nos. 3,507,277; 3,518,992; 3,635,219; 3,795,244; and 3,807,400, inhalation devices are disclosed having means for piercing or removing the top of a capsule containing a powdered medication which, upon inhalation, is drawn out of the pierced or topped capsule and into the user's mouth. Several of these patents disclose propeller means, which upon inhalation aid in dispensing the powder out of the capsule, so that it is not necessary to rely solely on the inhaled air to suction powder from the capsule. For example, in U.S. Pat. No. 2,517,482, a device is disclosed having a powder containing capsule placed in a lower chamber before inhalation, where it is pierced by manual depression of a piercing pin by the user. After piercing, inhalation is begun and the capsule is drawn into an upper chamber of the device where it moves about in all directions to cause a dispensing of powder through the pierced hole and into the inhaled air stream. U.S. Pat. No. 3,831,606 discloses an inhalation device having multiple piercing pins, propeller means, and a self-contained power source for operating the propeller means via external manual manipulation, so that upon inhalation the propeller means aids in dispensing the powder into the stream of inhaled air. See also U.S. Pat. No. 5,458,135.

These prior art devices present several problems and possess several disadvantages, which are remedied by the inhalation devices of the present invention. For one, these devices rely on additional mechanical components to pierce the blisters resulting in increased production costs. Also, these prior art devices require that the user exert considerable effort in inhalation to effect dispensing or withdrawal of powder from a pierced capsule into the inhaled air stream. With these prior art devices, suction of powder through the pierced holes in the capsule caused by inhalation generally does not withdraw all or even most of the powder out of the capsule, thus causing a waste of the medication. And, such prior art devices may result in uncontrolled amounts or clumps of powdered material being inhaled into the user's mouth, rather than a constant inhalation of controlled amounts of finely dispersed powder.

Another major drawback of the above mentioned multi unit-dose DPIs besides the complexity of piercing mechanisms, etc. is the inability to package large number of doses in the inhaler. The inability of the inhalers to package doses in excess of 50 dose in the inhaler puts these DPIs at a competitive disadvantage to MDIs (metered dose inhalers) which normally package in excess of 100 doses in the canister. U.S. Pat. No. 5,590,645 attempts to address this issue. U.S. Pat. No. 5,590,645 to Davies et al. describes an inhalation device for use with a blister pack which includes a flexible strip comprising a base strip in which a plurality of pockets or blisters for powdered medicament are formed, covered by a lid sheet peelably secured to the base strip. The device includes a lid winding wheel for peeling the strips apart to open the pocket or blister; and a manifold, communicating with the opened pocket or blister, through which a user can inhale medicament in powder form from the opened pocket or blister. However, the Davies et al. device and blister pack is somewhat complicated mechanically, and complete utilization of powdered medicament is not always possible due to the shape and depth of the pockets or blisters.

### SUMMARY OF THE INVENTION

The present invention provides an improved blister pack and inhaler device that overcomes the aforesaid and other disadvantages and drawbacks of the prior art. More particularly, the present invention provides an improved blister pack formed from a web or tape which is folded or pleated on itself to define a plurality of spaced pockets in which measured quantities of a pharmaceutical or drug may be loaded.

The invention also provides an inhaler for functioning with a blister pack formed from a folded or pleated web or tape in which the folds or pleats define a plurality of pockets in which a measured quantity of a pharmaceutical or drug is loaded.

### BRIEF DESCRIPTION OF THE DRAWINGS

Other features and advantages of the present invention will be apparent from the following description, taken in conjunction with the accompanying drawings, wherein like reference numerals designate like parts, and wherein:

FIG. 1 is a side elevational view of a blister pack made in accordance with the present invention;

FIG. 2 is a block flow diagram and FIGS. 3A-3C are perspective views illustrating the formation of a blister pack of the present invention;

FIG. 4 is a side elevational view, in partial cross-section, of a blister pack cartridge made in accordance with the present invention;

FIG. 5 is a side elevational view, in partial cross-section of an inhaler made in accordance with the present invention; and

FIG. 6 is a plan view of an alternative form of a blister pack made in accordance with the present invention.

### DETAILED DESCRIPTION

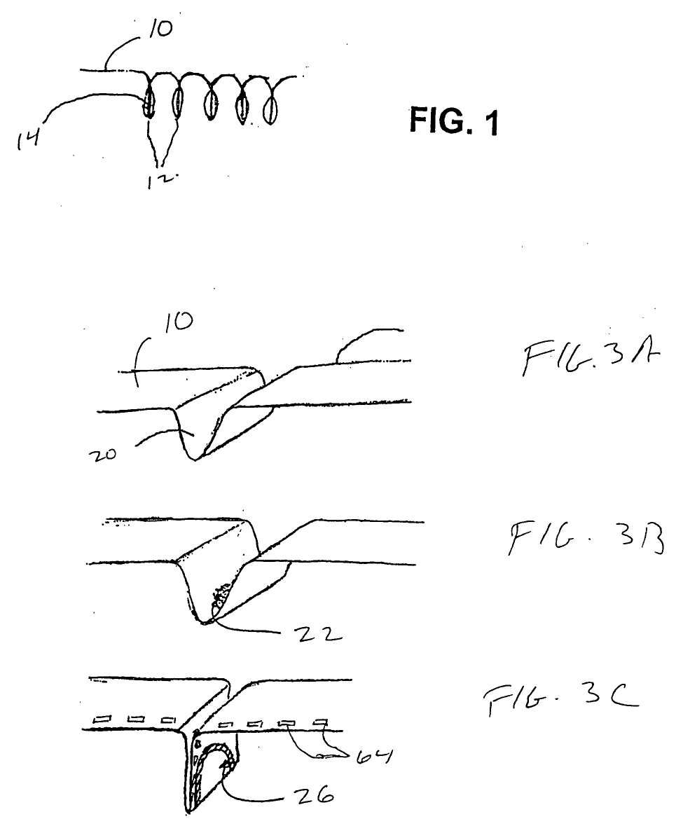

Referring to FIG. 1, a blister pack, in accordance with the present invention comprises an elongated web or tape 10 folded or pleated to form a plurality of folds or pleats 12 in which is loaded a measured quantity of a pharmaceutical or drug 14. Tape 10 is formed of a flexible material approved for contact with a pharmaceutical or drug. Preferably, tape 10 comprises a trilaminate of plastic film and aluminum foil to allow for good moisture protection.

Referring also to FIGS. 2 and 3A-3C, manufacture of a blister pack of FIG. 1 is quite straightforward. An elongated tape 10 is fed to a pleating station 16 wherein a pleat or pocket 20 is formed in the tape. A measured quantity 22 of a pharmaceutical or drug is then loaded into the pleat 20 at a loading station 24. The pleat or pocket 20 is then sealed at 26 around the pharmaceutical or drug at a sealing station 28. The sealing may be accomplished by mechanical means, for example, crimping, by use of an adhesive, or by heat or pressure welding. In a particularly preferred embodiment of the invention seal 26 is formed by using heat. The sealing pattern, amount of heat and the pressure applied is such as to provide a good seal while allowing for peelable separation.

A plurality of like pleats or pockets may be formed spaced apart from another by advancing the tape 10, and repeating steps 22, 26 and 28.

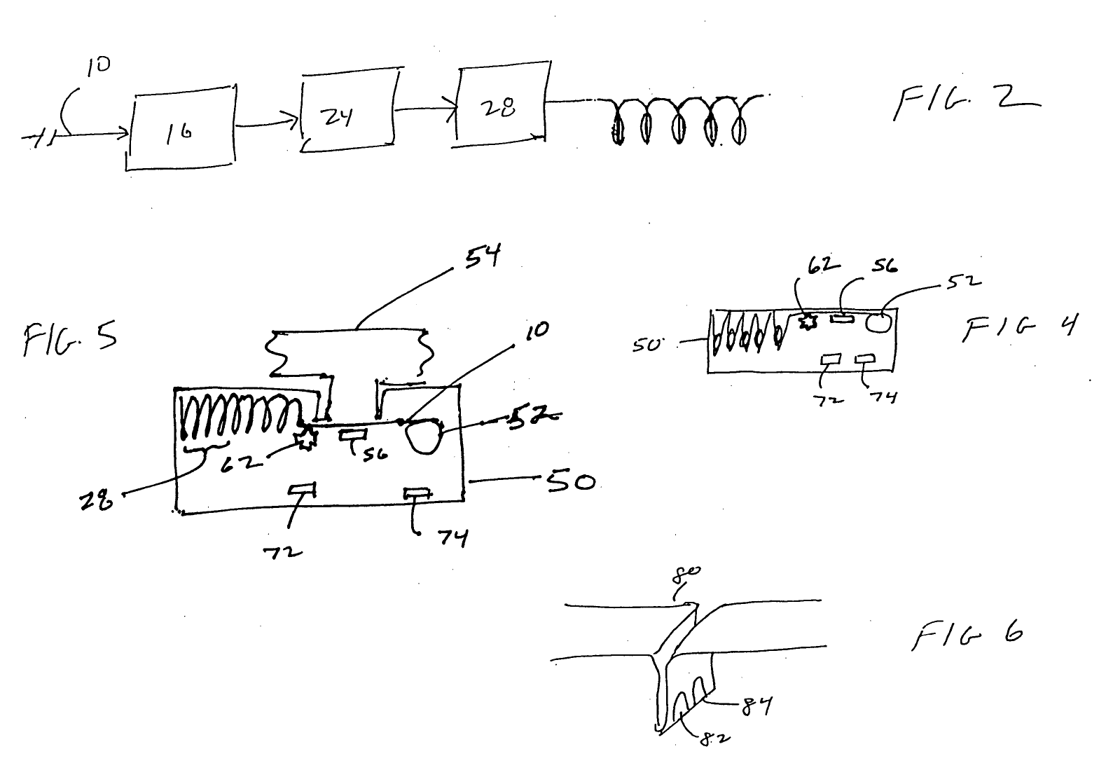

Referring to FIGS. 4 and 5, a tape having a plurality of pleats or pockets 28 is loaded accordion style into a cartridge 50. Cartridge 50 also includes a take-up reel 52 around which spent tape 10 may be wound. Cartridge 50 is loaded into an inhaler 54 which, in a preferred embodiment includes one or a plurality of vibratory or piezo elements 56, the purpose of which will be described in detail hereinafter.

Inhalation device 54 is similar to the inhalation device described in my earlier U.S. Pat. No. 6,026,809. However, rather than opening individual blisters by peeling back a film, individual pleats or pockets are opened by mechanically restraining or holding the tape to one side of a blister, and pulling the tape at other side of the pleat or pocket so that the pleat or pocket is pulled out and the tape flattened against the piezo elements 56. Accordingly, in place of the release film take-up spool of '809 patent, there is provided a means for selectively restraining or holding the tape. The holding means may comprise, for example a clamping means, detent or sprocket for indexing the tape so that an open blister will be positioned over the piezo element. In a preferred embodiment the inhaler includes a toothed sprocket wheel 62 for engaging sprocket holes 64 (see FIG. 3C) formed in an edge of the elongated tape. In use, the tape is advanced to position a fresh pleat or pocket over the top surface of the piezo element 56. The sprocket wheel 62 is then locked by means of a detent or shaft lock (not shown), and a take-up reel 52 on the far side of the piezo element 56 is advanced to pull the pleat or pocket open and flat against the piezo element 56.

The piezoelectric element 56 mechanically engages the bottom of the tape under the opened pleats or pockets as they are selectively advanced in position over and in contact with the piezoelectric element 56. The process of opening the pleats maximizes the surface area of the flattened tape in contact with the piezoelectric element 56, thus maximizing coupling of the tape with the piezoelectric element 56.

Piezoelectric element 56 is made of a material that has a high-frequency, and preferably, ultrasonic resonant vibratory frequency (e.g., about 15 to 50 kHz), and is caused to vibrate with a particular frequency and amplitude depending upon the frequency and/or amplitude of excitation electricity applied to the piezoelectric element 56. Examples of materials that can be used to comprise the piezoelectric element 56 include quartz and polycrystalline ceramic materials (e.g., barium titanate and lead zirconate titanate). Advantageously, by vibrating the piezoelectric element 56 at ultrasonic frequencies, the noise associated with vibrating the piezoelectric element 56 at lower (i.e., non-ultrasonic) frequencies can be avoided.

Maximum transfer of vibratory power from the piezoelectric element 56 to the powder in the open blister 20 takes place when the piezoelectric element 56 vibrates at its resonant frequency. It has been found that this results in maximum de-aggregation and suspension of the powder from the opened pleat into the air to be inhaled by the user. Preferably, the initial frequency and amplitude of actuating electricity supplied to the piezoelectric element 56 is pre-calibrated to cause the piezoelectric element 56 to vibrate at its resonance frequency when no opened pleat is present. However, when an opened pleat is placed against the piezoelectric element 56, the weight and tension of the tape, and the weight, volume, and particular size of the powder to be suspended by the piezoelectric element can change the vibration characteristics of the piezoelectric element, and cause the piezoelectric element to vibrate at other than its resonant frequency. Thus, a feedback control system similar to the feedback system described in my aforesaid U.S. Pat. No. 6,026,809 preferably is used to adjust vibration of the piezoelectric element to vibrate at its resonant frequency and maximize the transfer of power to the powder.

Alternatively, two piezoelectric elements can be used instead of one. When two piezoelectric elements are used, they may be designed to vibrate at different amplitudes and frequencies, i.e. so that, for example, two different drugs advantageously may be dispersed simultaneously from side-by-side pockets or folds in the same inhaler, without compromising performance or either drug. A tape 80 with side-by-side pockets 82, 84 made in accordance with the present invention is illustrated in FIG. 6. This permits delivery of two drugs which, while active together, may not readily be stored together. For example, an asthma inhaler may be provided containing both a bronchodilator, such as albuterol, and a steroid which may require different peizo settings.

Alternatively, the vibrator can be comprised of a magnetostriction device. A magnetostriction vibrator can be formed of a ferromagnetic material, such as nickel, that will cause the material to change dimensions in response to an induced magnetic flux.

Instead of a magnetostriction device or piezoelectric vibrator, other means to de-aggregate and aerosolize the dry powder may be used in alternative or in conjuncture with the aforementioned methods. For example, opposite electric or magnetic charges may be induced on the dry powder and parts of the inhaler to aerosolize the powder.

Finally, an actuating circuit indicated generally at 72 and a power supply such as a battery 74 are mounted within the cartridge 50. Alternatively, the power supply and activating circuit may be mounted within the inhalation device 60.

It should be emphasized that the above-described embodiments of the present invention are merely possible examples of implementations, merely set forth for a clear understanding of the principles of the invention. Many variations and modifications may be made to the above-described embodiments of the invention without departing from the spirit and principles of the invention. All such modifications and variations are intended to be included herein within the scope of this disclosure and the present invention and protected by the following claims.

1. A medication pack for use in an inhalation device comprising:
a sheet of material doubled onto itself to form a least one pleat having facing inside walls, wherein at least a portion of said facing inside walls are affixed to one another to form a pocket for medication.

2. The pack as claimed in claim 1, having a drug within said pocket.

3. The pack as claimed in claim 1, comprising a single sheet having a plurality of spaced pockets.

4. The pack as claimed in claim 1, where said single sheet is formed of a biocompatible material.

5. The pack as claimed in claim 3, where each of said pockets holds a measured quantity of a pharmaceutical.

6. The pack as claimed in claim 5, wherein the pharmaceutical comprises a dry powder.

7. A medication pack for use in an inhalation device comprising:
a single elongated tape folded into a plurality of pockets, wherein each said pocket contains a medication.

8. The pack as claimed in claim 7, wherein each pocket is sealed adjacent its edges.

9. The pack as claimed in claim 7, comprising a plurality of side-by-side pockets.

10. The pack as claimed in claim 8, wherein at least some of said plurality of pockets contain different medications.

11. The pack as claimed in claim 8, wherein edges of said pockets are sealed.

12. The pack as claimed in claim 11, wherein edges of said pockets are heat-sealed.

13. The pack as claimed in claim 11, wherein edges of said pockets are adhesively sealed.

14. The pack as claimed in claim 11, wherein said tape is a made of a trilaminate.

15. The pack as claimed in claim 14, wherein said tape comprises a trilaminate of plastic film and aluminum foil.

16. The pack as claimed in claim 7, comprising a plurality of pockets folded accordion style.

17. The pack as claimed in claim 7, wherein the medication comprises a dry powder.

18. A method of manufacturing a drug storage device comprising the steps of:
folding an elongated sheet to form a pocket;
depositing a drug in said pocket;
sealing edges of the pocket to contain the drug.

19. The method of manufacturing a drug storage device as claimed in claim 18, wherein the edges of said pocket are sealed using heat or an adhesive.

20. The method of manufacturing a drug storage device comprising the steps of:
providing an elongated pleated sheet;
disposing a drug within at least one of said pleats; and
sealing edges of the pleat to contain the drug.

21. An inhalation device for use with a medication pack as claimed in claim 7, said device comprising:
a chamber equipped with a mechanism for deaggregating said medication;
a mechanism for advancing each pocket to said chamber and for opening said pocket in said chamber; and
an airflow passage to carry deaggregated medication from said chamber.

22. The inhalation device as claimed in claim 21, wherein the medication pack is in the form of a wound roll.

23. The inhalation device as claimed in claim 21, wherein the medication pack is in the form of a pleated tape.

24. The inhalation device according to claim 23, wherein the medication pack is in the form of a cartridge.

25. The inhalation device according to claim 24, wherein the cartridge is disposable.

26. The blister pack according to claim 24, wherein the cartridge includes a power source.

27. The inhalation device according to claim 21, wherein the mechanism for deaggregating the medication comprises a vibrator.

28. The inhalation device according to claim 27, wherein the vibrator comprises a piezoelectric vibrator.

29. The inhalation device according to claim 28, comprising two or more piezoelectric vibrators.

30. An inhalation device for use with medication pack as claimed in claim 1, said device comprising:
a mechanism for receiving and opening the medication pack to expose the drug; and
an air-flow passageway to carry the drug to the patient.

31. An inhalation device for use with a medication pack as claimed in claim 7, said device comprising;
a mechanism for advancing each pocket and for opening said pocket to expose to expose medication to a chamber; and
an air-flow passageway in communication with said chamber to carry medication to the patient.
